(19) United States
(12) Patent Application Publication
(10) Pub. No.: US 2006/0234971 A1
(43) Pub. Date: Oct. 19, 2006

(54) Calpains as targets for inhibition of prion propagation

(75) Inventors: Glenn C. Telling, Lexington, KY (US); Rodney P. Guttman, Lexington, KY (US)

Correspondence Address: BUCHANAN, INGERSOLL & ROONEY PC, POST OFFICE BOX 1404, ALEXANDRIA, VA, 22313-1404, US

(73) Assignee: University of Kentucky, Lexington, KY (US)

(21) Appl. No.: 11/386,646

(22) Filed: Mar. 23, 2006

Related U.S. Application Data
(60) Provisional application No. 60/665,055, filed on Mar. 23, 2005.

(51) Int. Cl. A61K 48/00; A61K 38/54; A61K 39/395

(52) U.S. Cl. 514/44; 514/2; 424/146.1

(57) ABSTRACT

The present invention relates to methods for the inhibition of disease-associated prion formation and propagation. Such methods are based on inhibition of PrP^{Sc} cleavage, which prevents PrP^{Sc} accumulation and results in reduced prion titers. More particularly, the present invention relates to endoproteolytic cleavage of PrP^{Sc} by calpain, a calcium (Ca²⁺)-activated cysteine protease, and its inhibition.

### CROSS-REFERENCE TO RELATED APPLICATION

The application claims priority under 35 U.S.C. §119 to U.S. Provisional Application No. 60/665,055 filed Mar. 23, 2005, the entire content of which is hereby incorporated herein by reference.

### IDENTIFICATION OF FEDERAL FUNDING

The applicant was in receipt of Grants N01-AI-25491 and RO1 NSIA14O334 from the U.S. Public Health Service during the time the invention was developed, and therefore the government may have rights in the invention.

### TECHNICAL FIELD

The present invention relates to methods for the inhibition of disease-associated prion formation and propagation. Such methods are based on inhibition of PrP^{Sc} cleavage, which prevents PrP^{Sc} accumulation and results in reduced prion titers. More particularly, the present invention relates to the endoproteolytic cleavage of PrP^{Sc} by calpain, a calcium (Ca²⁺)-activated cysteine protease, and its inhibition.

### BACKGROUND OF THE INVENTION

Prion diseases are transmissible neurodegenerative disorders that include bovine spongiform encephalopathy (BSE), scrapie in sheep, chronic wasting disease (CWD) of deer and elk and human Creutzfeldt Jakob disease (CJD). While the detailed mechanism of prion propagation remains to be determined, considerable evidence suggests that prions are devoid of nucleic acid, and are composed largely, if not entirely, of the scrapie isoform of the prion protein (PrP), referred to as PrP^{Sc}. During the disease process, PrP^{Sc} acts as a template for conversion by imposing its conformation on the normally benign host-encoded version of the prion protein referred to as PrP^{C} (reviewed in Weissmann, C., Enari, M., Klohn, P. C., Rossi, D., and Flechsig, E. (2002) Proc Natl Acad Sci USA 99 Suppl 4, 16378-16383). The conversion of PrP^{C} into PrP^{Sc} involves a profound conformational change: PrP^{C} has a high α-helical content and is virtually devoid of β-sheets while PrP^{Sc} has a high β-sheet content (see, for example, Caughey, B. W., Dong, A., Bhat, K. S., Ernst, D., Hayes, S. F., and Caughey, W. S. (1991) Biochemistry 30, 7672-7680; Pan, K.-M., Baldwin, M., Nguyen, J., Gasset, M., Serban, A., Groth, D., Mehlhorn, I., Huang, Z., Fletterick, R. J., Cohen, F. E., and Prusiner, S. B. (1993) Proc. Natl. Acad. Sci. USA 90, 10962-10966; and Safar, J., Roller, P. P., Gajdusek, D. C., and Gibbs, C. J., Jr. (1993) J. Biol. Chem. 268, 20276-20284). A hallmark of PrP^{Sc} is its insolubility in non-denaturing detergents and its relative resistance to protease digestion in vitro. Proteinase K (PK) treatment of PrP^{Sc} results in the persistence of a core molecule, referred to as PrP27-30, consisting predominantly of amino acid residues 89 to 230 (mouse PrP residue numbering) (Oesch, B., Westaway, D., Wälchli, M., McKinley, M. P., Kent, S. B. H., Aebersold, R., Barry, R. A., Tempst, P., Teplow, D. B., Hood, L. E., Prusiner, S. B., and Weissmann, C. (1985) Cell 40, 735-746). In contrast to PrP^{Sc}, PrP^{C} is soluble in detergents and sensitive to proteolytic digestion by PK.

In addition to these biochemical differences, PrP^{C} and PrP^{Sc} are subject to diverse intracellular proteolytic processing events (Pan, K.-M., Stahl, N., and Prusiner, S. B. (1992) Protein Sci. 1, 1343-1352; Harris, D. A., Huber, M. T., van Dijken, P., Shyng, S.-L., Chait, B. T., and Wang, R. (1993) Biochemistry 32, 1009-1016; and Taraboulos, A., Scott, M., Semenov, A., Avrahami, D., Laszlo, L., and Prusiner, S. B. (1995) J. Cell Biol. 129, 121-132). Previous studies demonstrated that human PrP^{C} undergoes proteolytic cleavage at amino acids 110/111 within a segment of conserved hydrophobic amino acids to produce an ˜17 kDa carboxyl-terminal fragment referred to as C1, while a PK resistant fragment of PrP is produced in infected brains, apparently as a result of cleavage at the same location that PK cleaves PrP^{Sc} in vitro (following amino acid residue 88 in mouse PrP). The latter cleavage produces a carboxyl-terminal fragment, referred to as C2, with the same apparent molecular mass as unglycosylated PrP27-30 (Chen, S. G., Teplow, D. B., Parchi, P., Teller, J. K., Gambetti, P., and Autilio-Gambetti, L. (1995) J. Biol. Chem. 270, 19173-19180). While recent studies suggest that ADAM/TACE matrix metalloproteases may be responsible for the generation of the C1 fragment (Vincent, B., Paitel, E., Saftig, P., Frobert, Y., Hartmann, D., De Strooper, B., Grassi, J., Lopez-Perez, E., and Checler, F. (2001) J. Biol Chem 276, 37743-37746), the identity of the cellular protease responsible for endoproteolytic cleavage of PrP^{Sc} and the role of the C2 cleavage product in prion pathogenesis have not been explored.

The calpain family of proteolytic enzymes is comprised of ubiquitous and tissue-specific isoforms of Ca²⁺-activated cysteine proteases that modify the properties of substrate proteins by cleavage at a limited number of specific sites (Huang, Y., and Wang, K. K. (2001) Trends Mol Med 7, 355-362) generating large, often catalytically active fragments. The regulatory function of calpains is in contrast to the digestive functions of, for instance, the lysosomal proteases or the proteasome. Proteolysis by calpains is involved in a wide range of cellular functions, including cellular differentiation, integrin-mediated cell migration, cytoskeletal remodeling and apoptosis (reviewed in Goll, D. E., Thompson, V. F., Li, H., Wei, W., and Cong, J. (2003) Physiol Rev 83, 731-801). Calpains have also been implicated in a number of neurodegenerative diseases, including brain injury, Alzheimer's disease, Parkinson's disease and Huntington's disease (see, for example, Huang, Y., and Wang, K. K. (2001) Trends Mol Med 7, 355-362; Kim, Y. J., Yi, Y., Sapp, E., Wang, Y., Cuiffo, B., Kegel, K. B., Qin, Z. H., Aronin, N., and DiFiglia, M. (2001) Proc Natl Acad Sci USA 98, 12784-12789; and Mishizen-Eberz, A. J., Guttmann, R. P., Giasson, B. I., Day III, G. A., Hodara, R., Ischiropoulos, H., Lee, V. M.-Y., Trojanowski, J. Q., and Lynch, D. R. (2003) Journal of Neurochemistry 86, 836-847). Calpain activity is tightly regulated in vivo by Ca²⁺ and by the specific intracellular protein inhibitor calpastatin. The two ubiquitously expressed calpains are m-calpain and μ-calpain, which are heterodimers made up of a catalytic (˜80 kDa) and a common regulatory (˜30 kDa) subunit that require millimolar and micromolar Ca²⁺ concentrations, respectively, for activation. Transgenic mice, in which the gene for the calpain regulatory subunit was ablated, lacked detectable m- and μ-calpain activity and died at mid-gestation (Arthur, J. S., Elce, J. S., Hegadom, C., Williams, K., and Greer, P. A. (2000) Mol Cell Biol 20, 4474-4481).

Previous studies have identified several distinct classes of prion inhibitors, including substituted tricyclic derivatives, tetrapyrrole compounds, cysteine protease inhibitors, branched polyamines, and specific anti-PrP antibodies (reviewed in Supattapone, S., Nishina, K., and Rees, J. R. (2002) Biochem Pharmacol 63, 1383-1388). While the mode of action of blocking antibodies appears to involve prevention of PrP^{Sc} formation by binding to PrP^{C}, and branched polyamines bind to and denature PrP^{Sc} in acidic compartments, the mechanism of inhibition by other inhibitors of PrP^{Sc} formation is not well characterized.

The present invention is based, in part, on a better understanding of the role of proteolytic cleavage in prion pathogenesis, and provides for methods that are directed at inhibition of pathogenesis-associated PrP^{Sc} cleavage reactions.

### SUMMARY OF THE INVENTION

The present invention relates to methods of treating prion related diseases in a subject in need thereof, comprising administering to the subject a therapeutically effective amount of a calpain inhibitor. Examples of calpain inhibitors include small organic molecules, peptides, small interfering RNA's (siRNAs), proteins, and anti-calpain antibodies.

Other aspects of the present invention are described throughout the specification.

### BRIEF DESCRIPTION OF THE DRAWINGS

FIG. 1 depicts the endoproteolytic processing of PrP^{C} and PrP^{Sc}. More particularly, schematic depiction is shown of full-length PrP^{C} and PrP^{Sc} following removal of amino and carboxyl-terminal signal peptides as well as the location at which each isoform undergoes proteolytic cleavage to produce C1 and C2 fragments. The locations of the five amino-terminal octapeptide repeats, represented as shaded boxes, the locations of secondary structure elements determined from NMR spectroscopic analysis of recombinant PrP in the carboxyl-terminal section of PrP^{C}, and the locations of Asn-linked carbohydrate additions to PrP^{C} and PrP^{Sc} are indicated. The location of the binding epitope for Fab-D18 on full-length PrP^{C} and PrP^{Sc} and C1 and C2 is also shown, as well as the expected molecular weights of the C1 and C2 fragments.

FIG. 2 depicts PrP^{C} and PrP^{Sc} cleavage by cellular proteases. The positions of protein molecular weight markers are shown to the left of the immunoblots. The locations of full-length PrP, C2 and C1 fragments are also indicated.

a: analysis of endoproteolytic cleavage of PrP in brain homogenates of uninoculated CD-1 Swiss mice and clinically sick CD-1 Swiss mice inoculated with mouse-adapted RML scrapie prions.

b: analysis of endoproteolytic cleavage of PrP in detergent extracts from uninfected SMB-PS cells and prion infected SMB cells.

c: treatment of recombinant mouse PrP (Rec MoPrP) with PNGaseF or Prnp^{0/0} brain extract.

d: extraction of C1 and C2 from SMB detergent lysates in the presence of protease inhibitor cocktail, PMSF MDL28170, or calpain inhibitor IV (Calpain IV).

FIG. 3 depicts the kinetics of PrP^{Sc}, C1 and C2 production in brain extracts from mice infected with RML prions. The positions of protein molecular weight markers are shown to the left of the immunoblots. The locations of full-length PrP, C2 and C1 fragments are also indicated.

a: kinetics of full-length PrP, C1 and C2 accumulation.

b: kinetics of PrP27-30 accumulation.

c: kinetics of accumulation of deglycosylated, PK-resistant material. The positions of protein molecular weight markers are shown to the left of the immunoblots. The locations of full-length PrP, C2 and C1 fragments are also indicated.

FIG. 4 depicts the effects of treatment of prion-infected cells with inhibitors of cellular proteases.

a: detergent cell extracts were isolated from control DMSO treated SMB cells and SMB cells treated with Cathepsin inhibitor III (Cath. III), Cathepsin L inhibitor III (Cath. L III), Caspase inhibitor III (Casp. III), Caspase 3 inhibitor III (Casp. 3 III), MG132, lactacystin, MDL28170, calpeptin and calpain inhibitor IV (calpain IV). A representative immunoblot of an inhibitor treatment experiment is shown. The positions of protein molecular weight markers are shown to the left of the immunoblots. The locations of full-length PrP, C2 and C1 fragments are also indicated. Immunoblots were probed with antibodies against actin to confirm equal protein loading.

b: quantification of C1 and C2 production in SMB cells treated with various protease inhibitors. Apparent amounts (densitometric units) of C1 and C2 in inhibitor-treated cells are plotted as a percentage of C1 and C2 in control treated SMB cells in the same experiment. Mean values of triplicate measurements±standard deviations of the means are shown. Levels of C2 are represented by black filled bars, and levels of C1 we represented by grey filled bars.

c: treatment of SMB cells with MDL28170, calpeptin or calpain inhibitor IV (50 μM each) demonstrating that cell toxicity is not triggered.

FIG. 5 depicts dose-dependent inhibition of C2 and corresponding increase in C1 levels in SMB cells treated with calpain inhibitors.

a and c: representative immunoblots showing the effects of different concentrations of calpain inhibitor IV and MDL28170, respectively, on endoproteolytic cleavage of PrP in SMB cells are shown. The positions of protein molecular weight markers are shown to the left of the immunoblots. The locations of full-length PrP, C2 and C1 fragments are also indicated. Immunoblots were also probed with antibodies against actin to confirm equal protein loading.

b: quantification of C2 production in SMB cells following treatment with calpain inhibitor IV or MDL28170. C2 levels in calpain inhibitor IV treated cells are represented by filled circles, and C2 levels in MDL28170 treated cells are represented by open circles.

d: quantification of C1 production in SMB cells following treatment with calpain inhibitor IV or MDL28170. C1 levels in calpain inhibitor IV treated cells are represented by filled circles, and C1 levels in MDL28170 treated cells are represented by open circles. Apparent amounts (densitometric units) of C1 and C2 in inhibitor-treated cells were plotted as a percentage of amounts in control treated SMB cells. Mean values of triplicate measurements±standard deviations of the means are shown.

FIG. 6 depicts effects of calpastatin and Ca²⁺ ionophore ionomycin on C2 production.

a: stable over expression of calpastatin inhibits C2 production in SMB cells. Equivalent amounts of proteins on immunoblots were also probed with antibodies against calpastatin and actin.

b: the Ca²⁺ ionophore ionomycin facilitates calpain-mediated cleavage of PrP^{Sc} in the presence of Ca²⁺ resulting in increased C2 production.

c: levels of m- and μ-calpains in SMB-PS, SMB, N2A and ScN2A cells. The positions of protein molecular weight markers are shown to the left of the immunoblots. The locations of full-length PrP, C2 and C1 fragments are also indicated.

FIG. 7 depicts the inhibition of PrP^{Sc} accumulation and prion propagation by calpain inhibition with MDL281703. Protease-resistant PrP27-30 was purified from detergent cell extracts.

a: dose-dependent inhibition of PrP27-30 accumulation in SMB cells by MDL28170.

b: densitometric analysis of PrP27-30 accumulation in SMB cells treated for 8 days with various concentrations of MDL281703 in three separate experiments. Apparent amounts (densitometric units) of PrP27-30 in inhibitor-treated cells were plotted as a percentage of PrP27-30 in control treated SMB cells in the same experiment. Mean values of triplicate measurements±standard deviations of the means are shown.

c: inhibition of PrP^{Sc} production in ScN2A cells by MDL28170.

d: re-emergence of PrP^{Sc} in SMB cells after removal of MDL28170. SMB cells were continuously cultured in the presence (+) or absence (−) of MDL28170 for 5 passages, after which time inhibitor was removed and inhibitor- or control-treated cells were grown for an additional 5 passages in MDL28170-free medium with PrP27-30 purified from detergent extracts prepared at each passage (referred to as passages 1 through 5) and. The positions of protein molecular weight markers are shown to the left of the immunoblots.

e: calpain inhibition impedes prion replication in SMB cells. Groups of 12 CD-1 Swiss mice were inoculated intracerebrally with MDL28170-treated SMB cells, represented by filled circles, and non-MDL-treated control SMB cells, represented by open circles, suspended in PBS.

### DETAILED DESCRIPTION OF THE INVENTION

The present invention relates to methods for the inhibition of disease-associated prion formation and propagation. Such methods are based on inhibition of PrP^{Sc} cleavage, which prevents PrP^{Sc} accumulation and results in reduced prion titers. More particularly, the present invention relates to the endoproteolytic cleavage of PrP^{Sc} by calpain, a calcium (Ca²⁺)-activated cysteine protease, and its inhibition.

Prion proteins (PrPs) exist in two basic forms. The normal cellular form, PrP^{C}, and the abnormal disease-associated form, PrP^{Sc}. As discussed in more detail elsewhere herein, it has been shown that PrP^{C} undergoes cleavage into a 17 kDa C-terminal fragment designated C1, whereas PrP^{Sc} undergoes cleavage into a 21 kDa N-terminal fragment designated C2. It is the C2 fragment that has been shown to be associated with active prion infections. While increases in intracellular Ca²⁺ stimulate production of C2, calpain inhibition results in reduced C2 levels, less PrP^{Sc} accumulation and diminished prion titers. Accordingly, inhibition of calpain provides a new target for treatment of prion infections.

Definitions

To facilitate understanding of the invention set forth in the disclosure that follows, a number of terms are defined below.

The term “prion” refers generally to infectious proteins that lack nucleic acid and have been implicated as the cause of various neurodegenerative diseases (such as scrapie, Creutzfeldt-Jakob disease, and bovine spongiform encephalopathy.)

The term “PrP” refers to the prion protein.

The term “PrP^{c}” refers to the normal cellular prion protein.

The term “PrP^{Sc}” refers to the abnormal, or disease-associated prion protein.

The term “calpain” refers to non-lysosomal, calcium-activated neural cysteine proteases.

The term “calpain inhibitor” refers to a compound that inhibits the proteolytic action of calpain-I or calpain-II, or both. The term calpain inhibitors as used herein include those compounds having calpain inhibitory activity in addition to or independent of their other biological activities.

The meaning of other terminology used herein should be easily understood by someone of ordinary skill in the art.

Calpain Inhibitors

The present invention relates to the use of calpain inhibitors to treat prion infections. Such inhibitors may take a variety of different forms, such as small organic molecules, peptides, small interfering RNA's (siRNAs), proteins (such as calpastatin), and anti-calpain antibodies.

Calpain inhibitors may take on several formulations including dipeptides or larger multimers (see for example: Donkor, I. O., Korukonda, R., Huang, T. L., LeCour, L., Jr. (2003). Peptidyl aldehyde inhibitors of calpain incorporating P2-proline mimetics. Bioorg Med Chem Lett. 13(5):783-4.; Inoue J., Nakamura M., Cui, Y. S., Sakai, Y., Sakai, O., Hill, J. R., Wang, K. K., Yuen, P. W. (2003). Structure-activity relationship study and drug profile of N-(4-fluorophenylsulfonyl)-L-valyl-L-leucinal (SJA6017) as a potent calpain inhibitor. J Med Chem. 27;46(5):868-71; and Montero, A., Albericio, F., Royo, M., Herradon, B. (2004). Solid-phase combinatorial synthesis of peptide-biphenyl hybrids as calpain inhibitors. Org Lett. 6(22):4089-92) as well as other organic compounds (see for example: Nakamura, M., Miyashita, H., Yamaguchi, M., Shirasaki, Y., Nakamura, Y., Inoue, J. (2003). Novel 6-hydroxy-3-morpholinones as cornea permeable calpain inhibitors. Bioorg Med Chem. 11(24):5449-60). Calpain activity is also inhibited by administration of calpain antibodies, a technique that has been previously shown to inhibit other enzymatic processes.

A wide variety of compounds have been demonstrated to have activity in inhibiting the proteolytic action of calpains. Examples of calpain inhibitors that are useful in the practice of the invention include N-acetyl-leucyl-leucylmethional (ALLM or calpain inhibitor II), N-acetyl-leucyl-leucyl-norleucinal (ALLN or calpain inhibitor 1), calpain inhibitor III (carbobenzoxy-valyl-phenylalanal; Z-Val-Phe-CHO), calpain inhibitor IV (Z-LLY-FMK; Z-LLY-CH₂ F where Z=benzyloxycarbonyl), calpain inhibitor V (Mu-Val-HPh-FMK where Mu is morphlinoureidyl and Hph is homophenylalanyl), calpeptin (benzyloxycarbonyldipeptidyl aldehyde; Z-Leu-Nle-CHO), calpain inhibitor peptide (Sigma No. C9181), calpastatin, acetyl-calpastatin (acetyl calpain inhibitor fragment, 184-210), leupeptin, mimetics thereof and combinations there, AK275, MDL28170 and E64. Additional calpain inhibitors are described in the following U.S. patents, incorporated herein by reference, U.S. Pat. Nos. 5,716,980; 5,714,471; 5,693,617; 5,691,368; 5,679,680; 5,663,294, 5,661,150; 5,658,906; 5,654,146; 5,639,783; 5,635,178; 5,629,165; 5,622,981; 5,622,967; 5,621,101; 5,554,767; 5,550,108; 5,541,290; 5,506,243; 5,498,728; 5,498,616; 5,461,146; 5,444,042; 5,424,325; 5,422,359; 5,416,117; 5,395,958; 5,340,922; 5,336,783; 5,328,909; 5,135,916.

Calpain inhibitors are commercially available. Exemplary protein calpain inhibitors are MDL28170, calpeptin and calpain inhibitor IV. Other suitable calpain inhibitors are listed in the following tables.

TABLE I
Calpain Inhibitors

| Product | Company | Catalog # |
|---|---|---|
| Calpastatin, human erythrocytes | Calbiochem | 208901 |
| Calpastatin, human, recombinant | Calbiochem | 208900 |
| Acetyl-Calpastatin, Acetyl Calpain | Sigma | C4285 |
| Inhibitor fragment, 184-210 | | |
| Ac-D-P-M-S-S-T-Y-I-E-E-L-G-K-R- | | |
| E-V-T-I-P-P-K-Y-R-E-L-L-A-NH₂ | | |
| Calpain Inhibitor Peptide D-P-M-S- | Sigma | C9181 |
| S-T-Y-I-E-E-L-G-K-R-E-V-T-I-P-P-K- | | |
| Y-R-E-L-L-A | | |
| Calpain Inhibitor I | Roche | 1 086 090 |
| N-acetyl-L-L-norleucinal | BioMol | P-120 |
| ALLN | Fluka | 21277 |
| | Sigma | A6185 |
| | Calbiochem | 208719 |
| Calpain Inhibitor II | Roche | 1 086 103 |
| N-acetyl-L-L-methional | Fluka | 21278 |
| ALLM | Calbiochem | 208721 |
| | Sigma | A6060 |
| | BioMol | PI-100 |
| Calpain Inhibitor III | Calbiochem | 208722 |
| carbobenzoxy-valyl-phenylalanal | | |
| MDL #28170 | | |
| Z-Val-Phe-CHO | | |
| (Z = benzyloxycarbonyl) | | |
| Calpain Inhibitor IV | Calbiochem | 208724 |
| Z-LLY-FMK | | |
| Z-L-L-Y-CH₂F | | |
| (Z = benzyloxycarbonyl) | | |
| Calpain Inhibitor V | Calbiochem | 208726 |
| Mu-Val-HPh-FMK | | |
| (Mu = morphlinoureidyl) | | |
| (HPh = homophenylalanyl) | | |
| Calpeptin | BioMol | PI-101 |
| benzyloxycarbonyldipeptidyl aldehyde | Calbiochem | 03-34-0051 |
| Z-Leu-Nle-CHO | | |
| (Z = benzyloxycarbonyl) | | |
| trans-Epoxy succinyl-L-leucylamido-(4- | BioMol | PI-105 |
| guanidino) butane | | |
| Z-Leu-Leu-CHO | BioMol | PI-116 |
| MDL-28170 | BioMol | PI-130 |

TABLE 2
Calpain Antibodies

| | | |
|---|---|---|
| Product | Company | Catalog # |

μ-Calpain, large subunit

| | | |
|---|---|---|
| Anti-μ-Calpain, 80kDa | Affinity | MA3-940 |
| subunit, Clone 9A4H8D3, | Bioreagents | |
| mouse | BioMol | SA-257 |
| Anti-μ-Calpain, 80kDa | Affinity | MA3-941 |
| subunit, Clone 2H2A7C2, | Bioreagents | |
| mouse | BioMol | SA-256 |
| Anti-μ-Calpain, 80kDa | Research | RDI-UCALPAINabm |
| subunit, PC-6, mouse | Diagnostics, Inc | |
| Anti-μ-Calpain, 80kDa | Research | RDI-CALPN1CabG |
| subunit, goat | Diagnostics, Inc | |
| Anti-μ-Calpain, 80kDa | Research | RDI-CALPN1NabG |
| subunit, goat | Diagnostics, Inc | |
| Anti-μ-Calpain, 80kDa | Triple Point | RP1CALPAIN1 |
| subunit, rabbit, domain I | Biologics | |
| Anti-μ-Calpain, 80kDa | Triple Point | RP2CALPAIN1 |
| subunit, rabbit, domain I | Biologics | |
| Anti-μ-Calpain, 80kDa | Triple Point | RP3CALPAIN1 |
| subunit, rabbit, domain IV | Biologics | |
| Anti-μ-Calpain, 80kDa | Triple Point | RP4CALPAIN1 |
| subunit, rabbit, domain IV | Biologics | |

m-Calpain, large subunit

| | | |
|---|---|---|
| Anti-m-Calpain, 80kDa | Affinity | MA3-942 |
| subunit, Clone 107-82, | Bioreagents | |
| mouse | BioMol | SA-255 |
| Anti-m-Calpain, 80kDa | Research | RDI-MCALPAINabr |
| subunit, PC1, rabbit | Diagnostics, Inc | |
| Anti-m-Calpain, 80kDa | Research | RDI-CALPN2NabG |
| subunit, goat | Diagnostics, Inc | |
| Anti-m-Calpain, 80kDa | Triple Point | RP1CALPAIN2 |
| subunit, rabbit, domain III | Biologics | |
| Anti-m-Calpain, 80kDa | Triple Point | RP2CALPAIN2 |
| subunit, rabbit, domain I | Biologics | |
| Anti-m-Calpain, 80kDa | Triple Point | RP3CALPAIN2 |
| subunit, rabbit, domain IV | Biologics | |
| Anti-m-Calpain, 80kDa | Triple Point | RP4CALPAIN2 |
| subunit, rabbit, domain III | Biologics | |

Calpain, small subunit

| | | |
|---|---|---|
| Anti-Calpain, 28kDa | Affinity | MA3-943 |
| subunit, Clone 156, mouse | Bioreagents | |
| Anti-Calpain, 28kDa | Research | RDI-CALPRGCabG |
| subunit, goat | Diagnostics, Inc | |
| Anti-Calpain, 28kDa | Research | RDI-CALPRGIabG |
| subunit, goat | Diagnostics, Inc | |

Calpain 3 (p94)

| | | |
|---|---|---|
| Anti-Calpain 3, rabbit, | Triple Point | RP1CALPAIN3 |
| Insert I | Biologics | |
| Anti-Calpain 3, rabbit, | Triple Point | RP2CALPAIN3 |
| Insert II | Biologics | |
| Anti-Calpain 3, rabbit, | Triple Point | RP3CALPAIN3 |
| domain III | Biologics | |
| Anti-Calpain 3, rabbit, | Triple Point | RP4CALPAIN3 |
| domain I | Biologics | |

Calpain 3 (Lp82/85)

| | | |
|---|---|---|
| Anti-Lp85, rabbit, | Triple Point | RP1LP85CALPAIN |
| domain IV | Biologics | |
| Anti-Lp82/85, rabbit, | Triple Point | RP1LP82/85CALPAIN |
| domain III | Biologics | |

TABLE 3
Calpastatin

| | | |
|---|---|---|
| Anti-Calpastatin, Clone | Affinity Bioreagents | MA3-944 |
| 1F7E3D10, mouse | BioMol | SA-284 |
| Anti-Calpastatin, Clone | Affinity Bioreagents | MA3-945 |
| 2G11D6, mouse | BioMol | SA-283 |

In an exemplary embodiment, the invention includes:

a. active site directed inhibitors such as:
MDL 28170
Calpain Inhibitor IV
Calpeptin
SJA6017, N-(4-fluorophenylsulfonyl)-L-valyl-L-leucinal
AK295,Z-Leu-aminobutyric acid-CONH(CH₂)₃-morpholine; Z=benzyloxycarbonyl
AK275, Z-Leu-Abu-CONH-CH2CH3; (Abu=χ-aminobutyric acid)
Z=benzyloxycarbonyl

b. calpastatin or calpastatin mimetics such as
CS 27-mer peptide (Calpain Inhibitor Peptide—amino acid sequence of: D-P-M-S-S-T-Y-I-E-E-L-G-K-R-E-V-T-I-P-P-K-Y-R-E-L-L-A).

c. compounds that bind to the calpain calcium binding domain such as:
PD 150606, [3-(4-Iodophenyl)-2-mercapto-(benzyloxycarbonyl)-2-propenoic acid]
PD 1I51746, 3-(5-fluoro-3-indolyl)-2-mercapto-(benzyloxycarbonyl)-2-propenoic acid

d. RNAi against calpain small subunit.

Calpain Targets

Based on the present findings, PrP^{Sc} propagation is initiated or enhanced by the action of endoproteolytic processing due to the activity of calpains. Thus, compounds that prevent calpain from generating C2 by: 1) interactions with calpain's active site cysteine (see for example, Hosfield, C. M., Elce, J. S., Jia, Z. (2004). Activation of calpain by Ca2+: roles of the large subunit N-terminal and domain III-IV linker peptides. J Mol Biol. 343(4): 1049-53.; Pal, G. P., De Veyra, T., Elce, J. S., Jia, Z. (2003). Crystal structure of a micro-like calpain reveals a partially activated conformation with low Ca2+ requirement. Structure (Camb). (12):1521-6; Hosfield, C. M., Elce, J. S., Davies, P. L., Jia, Z. (1999). Crystal structure of calpain reveals the structural basis for Ca(2+)-dependent protease activity and a novel mode of enzyme activation. EMBO J. 18(24):6880-9; Arthur, J. S., Gauthier, S., Elce, J. S. (1995).

Active site residues in m-calpain: identification by site-directed mutagenesis. FEBS Lett. 368(3):397-400; and Tompa, P., Buzder-Lantos, P., Tantos, A., Farkas, A., Szilagyi, A., Banoczi, Z., Hudecz, F., Friedrich, P. (2004). On the sequential determinants of calpain cleavage. J Biol Chem. 279(20):20775-85),or the additional two amino acids histidine or asparagines of the catalytic triad (see for example, Arthur, J. S., Elce, J. S. (1996).

Interaction of aspartic acid-104 and proline-287 with the active site of m-calpain. Biochem J.319 (Pt 2):535-41 and Berti, P. J., Storer, A. C. (1995). Alignment/phylogeny of the papain superfamily of cysteine proteases. J Mol Biol. 246(2):273-83); interactions with calpain's substrate binding areas, (see for example, Todd, B., Moore, D., Deivanayagam, C. C., Lin, G. D., Chattopadhyay, D., Maki, M., Wang, K. K., Narayana, S. V. (2003). A structural model for the inhibition of calpain by calpastatin: crystal structures of the native domain VI of calpain and its complexes with calpastatin peptide and a small molecule inhibitor. J Mol Biol. 328(1):131-46; Lin, G. D., Chattopadhyay, D., Maki, M., Wang, K. K., Carson, M., Jin, L., Yuen, P. W., Takano, E., Hatanaka, M., DeLucas, L. J., Narayana, S. V. (1997).

Crystal structure of calcium bound domain VI of calpain at 1.9 a resolution and its role in enzyme assembly, regulation, and inhibitor binding. Nat Struct Biol. 4(7):539-47; Mucsi, Z., Hudecz, F., Hollosi, M., Tompa, P., Friedrich, P. (2003). Binding-induced folding transitions in calpastatin subdomains A and C. Protein Sci. 12(10):2327-36; Todd, B., Moore, D., Deivanayagam, C. C., Lin, G. D., Chattopadhyay, D., Maki, M., Wang, K. K., Narayana, S. V. (2003). A structural model for the inhibition of calpain by calpastatin: crystal structures of the native domain VI of calpain and its complexes with calpastatin peptide and a small molecule inhibitor. J Mol Biol. 328(1): 131-46; Betts, R., Weinsheimer, S., Blouse, G. E., Anagli, J. (2003). Structural determinants of the calpain inhibitory activity of calpastatin peptide B27-WT. J Biol Chem. 278(10):7800-9; Takano, E., Ma, H., Yang, H. Q., Maki, M, Hatanaka, M.(1995). Preference of calcium-dependent interactions between calmodulin-like domains of calpain and calpastatin subdomains. FEBS Lett. 362(1):93-7; Croall, D. E., McGrody, K. S. (1994). Domain structure of calpain: mapping the binding site for calpastatin. Biochemistry. 33(45):13223-30; Ma, H., Yang, H. Q., Takano, E., Hatanaka, M., Maki, M. (1994).

Amino-terminal conserved region in proteinase inhibitor domain of calpastatin potentiates its calpain inhibitory activity by interacting with calmodulin-like domain of the proteinase. J Biol Chem. 269(39):24430-6; Crawford, C., Brown, N. R., Willis, A. C. (1993). Studies of the active site of m-calpain and the interaction with calpastatin. Biochem J. 296 (Pt 1):135-42; Kawasaki, H., Emori, Y., Suzuki, K. (1993).

Calpastatin has two distinct sites for interaction with calpain-effect of calpastatin fragments on the binding of calpain to membranes. Arch Biochem Biophys. 305(2):467-72;and Nishimura, T., Goll, D. E. (1991). Binding of calpain fragments to calpastatin. J Biol Chem. 266(18):11842-50); increasing calpastatin levels (see for example, Averna, M., De Tullio, R., Capini, P., Salamino, F., Pontremoli, S., Melloni, E. (2003).

Changes in calpastatin localization and expression during calpain activation: a new mechanism for the regulation of intracellular Ca(2+)-dependent proteolysis. Cell Mol Life Sci. 60(12):2669-78; Maekawa, A., Lee, J. K., Nagaya, T., Kamiya, K., Yasui, K., Horiba, M., Miwa, K., Uzzaman, M., Maki, M., Ueda, Y., Kodama, I. (2003).

Overexpression of calpastatin by gene transfer prevents troponin I degradation and ameliorates contractile dysfunction in rat hearts subjected to ischemia/reperfusion. J Mol Cell Cardiol. 35(10): 1277-84; and Guttmann, R. P., Sokol, S., Baker, D. L., Simpkins, K. L., Dong, Y., Lynch, D. R. (2002). Proteolysis of the N-methyl-d-aspartate receptor by calpain in situ. J Pharmacol Exp Ther. 302(3):1023-30, would be expected to inhibit prion propagation.

All such exemplary embodiments have been shown to reduce prion protein titre.

### EXAMPLES

### Experimental Procedures

Chemicals and Antibodies

For immunologic detection of PrP^{C} and PrP^{Sc}, recombinant PrP specific FAB D-18 was used (Peretz, D. et al., (2001) Nature 412:739-743). As described, FAB D-18 detects an epitope between amino acid residues 135-157, and therefore recognizes PrP^{C}, PrP^{Sc}, C1 and C2.

All immunoblots probed with Fab D-18 were developed using horse raddish peroxidase (HRP)-conjugated goat anti-Hu secondary antibody and ECL or ECL-Plus detection (Amersham Biosciences, Piscataway, N.J.) and exposed to x-ray film. Anti-calpastatin and anti-actin antibodies were purchased from Chemicon International, Inc., Temecula, Calif. All protease inhibitors were purchased from Calbiochem, EMD Biosciences, Inc., San Diego, Calif. Ionomycin and A23187 were purchased from Sigma-Aldrich Corp., St. Louis, Mo.

Cell Culture and Pharmacologic Treatments

Scrapie infected mouse brain (SMB) cells (Clarke, M. C., and Haig, D. A. (1970) Nature 225, 100-101), SMB-PS cells cleared of infectivity by pentosan sulfate (PS) treatment (Birkett, C. R., Hennion, R. M., Bembridge, D. A., Clarke, M. C., Chree, A., Bruce, M. E., and Bostock, C. J. (2001) Embo J 20, 3351-3358), neuro2A neuroblastoma cells (N2A) (obtained from the American Type Culture Collection, Manassas, Va.) and ScN2A which are a highly susceptible sub-line of N2A cells persistently infected with mouse-adapted scrapie RML prions (Bosque, P. J., and Prusiner, S. B. (2000) J Virol 74, 4377-4386) were maintained in Dulbecco's modified Eagle's medium supplemented with 10% fetal calf serum, penicillin (100 U/ml) and streptomycin (100 mg/ml) (Invitrogen Corporation, Carlsbad, Calif.) at 37° C. in 5% CO₂. SMB and SMB-PS cells were routinely sub-cultured 1:6 every 4 days and ScN2A cells were split 1:10 using 0.05% (w/v) trypsin-EDTA (Invitrogen Corporation, Carlsbad, Calif.). For inhibitor studies 0.75×10⁶ SMB cells were seeded on 6 cm dishes and 1.6×10⁶ SMB cells were seeded on 10 cm dishes. Stock solutions of protease inhibitors in dimethyl sulfoxide (DMSO) were added to cell culture media at various final concentrations. During inhibitor treatments, medium containing fresh inhibitor was replaced daily and control treated cells were cultured in medium containing an equal volume of DMSO without inhibitor. When cells achieved confluence, usually after 4 days, cells were lysed and total protein content was determined. For Ca²⁺ ionophore treatments, the culture medium of sub-confluent monolayers of SMB cells was replaced for 1 hour with Optimem (Invitrogen Corporation, Carlsbad, Calif.) containing 2 mM CaCl₂ and various concentrations of ionomycin or A23187, after which detergent extracts were prepared. In transfection experiments, SMB cells were transfected at 90% confluence with 10 μg of a modified version of the pRK5 expression plasmid containing a selectable neomycin resistance marker and the full-length human calpastatin cDNA using Lipofectamine 2000 (Invitrogen Corporation, Carlsbad, Calif.). Control cultures were transfected with empty vector expressing only the neomycin resistance gene. Transfected cultures were bulk selected in the presence of 0.35 mg/ml G418.

Analysis of PrP

Brain homogenates (10% (w/v) in phosphate buffered saline (PBS)) from RML scrapie-infected CD-1 Swiss mice and uninoculated CD-1 Swiss mice were prepared by repeated extrusion through an 18-gauge syringe needle followed by a 21-gauge needle in PBS lacking calcium and magnesium ions. Nuclei and debris were removed from brain homogenates by brief centrifugation at 1,000-×g. Sarkosyl was added at a final concentration of 2%. For cell cultures, after washing in PBS, cells were treated with cold lysis buffer (50 mM Tris pH 8.0, 150 mM NaCl, 0.5% Na deoxycholate, 0.5% IGEPAL CA-630) and cell debris was removed by centrifugation at 3,000-×g for 5 min. Total protein content of cell culture or brain extracts was determined by bicinchoninic acid (BCA) assay (Pierce Biotechnology, Inc., Rockford, Ill.) using a BioTek plate reader. For deglycosylation, 50 μg protein aliquots were mixed with recombinant PNGase F for 1 hour at 37° C., as specified by the supplier (New England Biolabs, Beverly, Mass.). In all experiments involving PK digestion, the final concentration of PK was 20 μg/ml and the ratio of total protein to PK was 50:1. Samples were incubated for 1 h at 37° C. and digestion was terminated by the addition of phenyl methyl sulfonyl fluoride (PMSF) to a final concentration of 2 mM. For PrP27-30 analysis in brain extracts, homogenates containing 50 μg total proteins were treated with PK. For PrP27-30 analysis in cultured cells, detergent extracts containing 1 mg total proteins in the case of SMB cells, or 2 mg total proteins in the case of ScN2A cells, were PK-treated and insoluble PrP27-30 was purified by ultracentrifugation for 1 hour at 100,000-×g in a Beckman TLX100 ultracentrifuge. Samples were boiled in an equal volume of 2× non-reducing SDS loading buffer for 5 minutes and resolved by SDS-PAGE. Proteins were transferred to polyvinylidene fluoride (PVDF) membranes and blocked with 5% (w/v) non-fat milk in Tris buffered saline containing Tween 20.

Cell Viability

0.2×10⁶ SMB cells were seeded in 6-well plates in medium containing 50 μM of MDL28170, calpeptin or calpain inhibitor IV. Medium containing fresh inhibitor was changed daily and control treated cells were cultured in medium containing an equal volume of DMSO without inhibitor. When cells achieved confluence they were trypsinized and re-seeded onto 6-well plates and the following day inhibitor-containing medium was replaced with normal medium containing 1 μg/ml calcein-AM and propidium iodide dyes for 30 minutes at 37° C. Medium was removed and cells were washed briefly with PBS. Cells were observed under 10× magnification using an Olympus IX 50 inverted florescent microscope. Counts of live cells fluorescing green and dead cells fluorescing orange were determined in a total of 4 fields for each well with a minimum of 200 cells counted in each field. Analysis was performed in triplicate for each inhibitor and the average and standard deviations were calculated for each condition.

Bioassay

The RML mouse scrapie prion isolate from Swiss mice was passaged in Swiss CD-1 mice obtained from Charles River Laboratories (Wilmington, Mass.). For inoculation of Swiss CD-1 mice, 10% (w/v) homogenates of RML-infected mouse brain were prepared by repeated extrusion through an 18-gauge syringe needle followed by a 21-gauge needle in PBS lacking calcium and magnesium ions. Samples were diluted 10-fold in PBS prior to inoculation. Mice were anaesthetized with a mixture of halothane and O₂, and inoculated intracerebrally with 30 μl of samples prepared from brain using a 27-gauge needle inserted into the right parietal lobe. All mice were thereafter examined thrice weekly for clinical signs of prion disease. As soon as any animal was identified as having progressive neurological symptoms consistent with prion infection, the animal was humanely killed by asphyxiation with CO₂. For bioassay of prion infectivity in MDL21870-treated and non-treated SMB cells, groups of CD-1 Swiss mice (n=12) were inoculated intracerebrally with MDL 28170-treated and control-treated SMB cells passaged in parallel. Cells were suspended in 1 ml of PBS and 30 μl cell suspension (˜1.8×10⁴ cells) was inoculated in each case into the right parietal lobe using a 27-gauge needle. The endpoint of the bioassay was the time to appearance of definitive clinical symptoms, referred to as the scrapie incubation time.

Quantification of PrP and Statistical Analyses

Densitometric analysis of C1, C2 and PrP27-30 levels in SMB cells was performed with a Kodak Imaging System using Image for Windows version 3b (Scion). All statistical analyses including student t tests were performed using GraphPad Prism version 4.0 for Windows, GraphPad Software, San Diego Calif. USA, www.graphpad.com.

### Results

PrP^{C} and PrP^{Sc} Cleavage by Endogenous Proteases in Brain and Cultured Cells

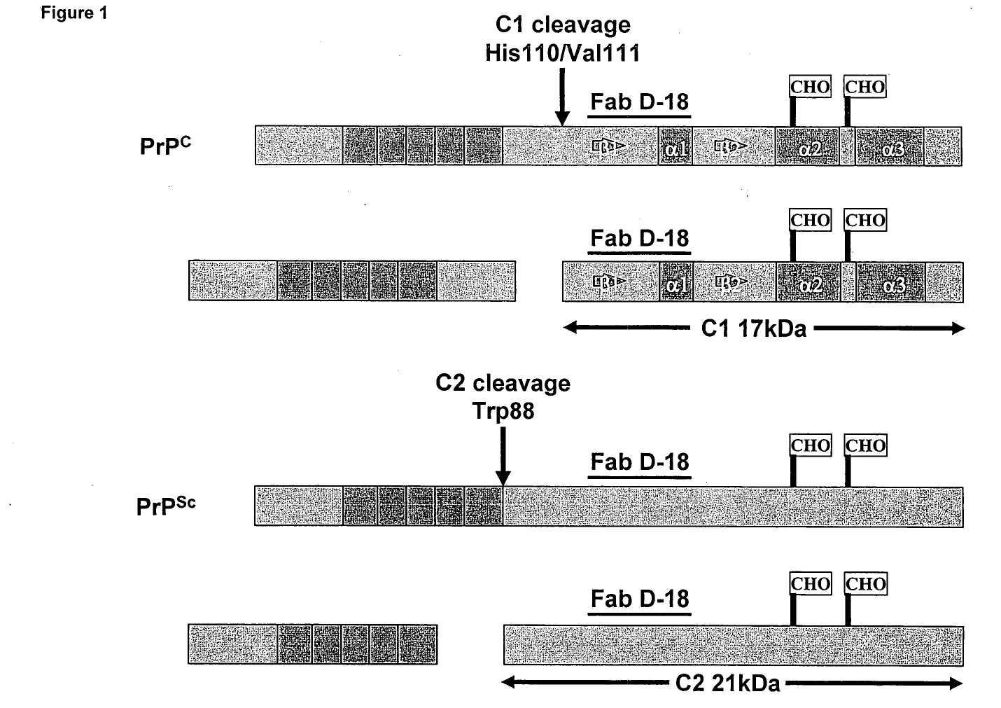

While PrP^{Sc} is usually defined by its relative resistance to protease digestion in vitro, the use of PK was avoided in this analyses to allow the detection of intact and endogenously cleaved forms of PrP^{C} and PrP^{Sc}. Since the C1 and C2 cleavage products contain the sites for asparagine (Asn)-linked glycosylation of PrP (FIG. 1) and, like full-length PrP, consist of multiple glycoforms that are normally obscured by other glycosylated and unglycosylated PrP species, Asn-linked glycans were removed by treatment of mouse brain homogenates and cultured cell extracts with PNGase F to simplify the analysis of PrP^{C} and PrP^{Sc} processing. For immunologic detection of mouse PrP, FAB D-18 was used.

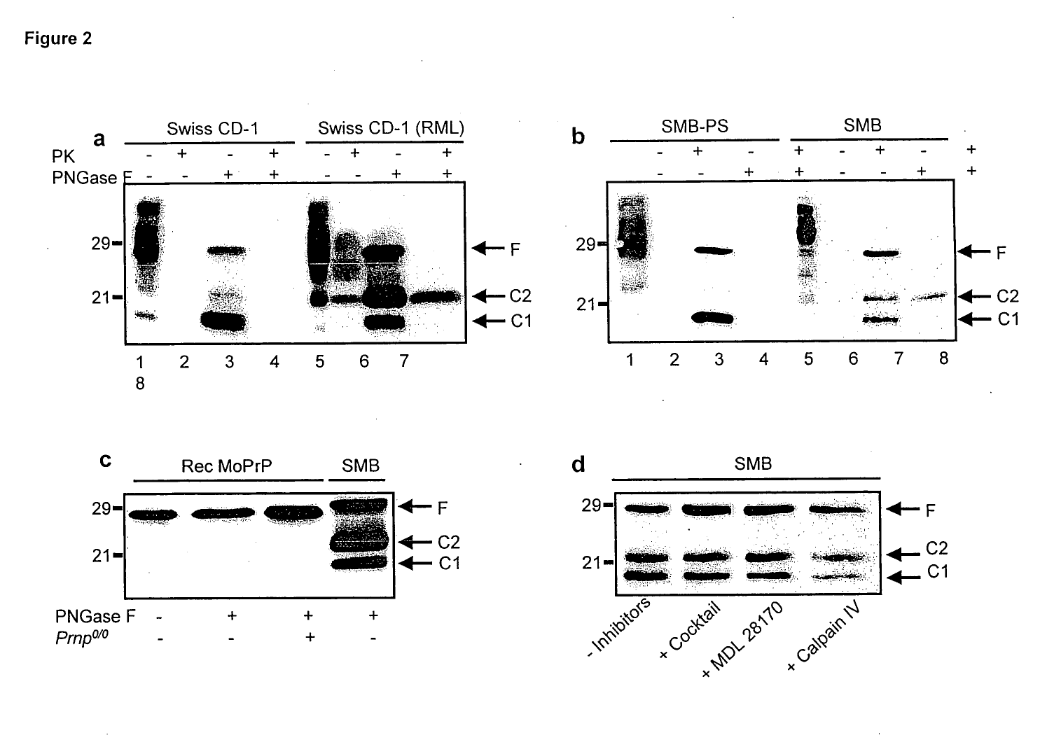

In addition to full-length PrP (F) with an apparent molecular weight of ˜28 kDa, carboxyl-terminal PrP fragments of 21 kDa and ˜17 kDa were detected in the brains of clinically affected mice infected with mouse-adapted RML scrapie prions (FIG. 2a, lane 7) while full-length PrP and the 17 kDa fragment predominated in uninfected mouse brains (FIG. 2a, lane 3). Like PrP^{Sc}, the 21 kDa fragment was partially resistant to PK and had the same apparent molecular weight as the unglycosylated form of as PrP27-30 (FIG. 2a, lanes 6 and 8). The 21 kDa fragment therefore corresponds mainly to the PrP^{Sc}-specific cleavage product, designated C2, detected in post mortem brain extracts from patients with CJD (Chen, S. G., Teplow, D. B., Parchi, P., Teller, J. K., Gambetti, P., and Autilio-Gambetti, L. (1995) J. Biol. Chem. 270, 19173-19180, Jimenez-Huete, A., Lievens, P. M., Vidal, R., Piccardo, P., Ghetti, B., Tagliavini, F., Frangione, B., and Prelli, F. (1998) Am J Pathol 153, 1561-1572). C2 was also detected in glycosidase-treated brain extracts from Syrian hamsters infected with the Sc237 strain of prions (K. Nishina and S. Supattapone, personal communication). The ˜17 kDa PK sensitive C-terminal PrP fragment, present in uninfected and RML infected mouse brain extracts (FIG. 2a), corresponds to the PrP^{C}-specific cleavage product C1 fragment previously identified in human brain extracts (Chen, S. G., Teplow, D. B., Parchi, P., Teller, J. K., Gambetti, P., and Autilio-Gambetti, L. (1995) J. Biol. Chem. 270, 19173-19180). While C2 was predominantly produced under conditions of prion infection, a cleavage product of similar molecular mass as C2 which did not survive treatment with PK was produced at much lower levels in uninfected mouse brains (FIG. 2a, lane 3). A similar endoproteolytically-cleaved fragment of human PrP, referred to as soluble PrP27-30, was detected in previous studies following partial deglycosylation of human platelet material (Perini, F., Vidal, R., Ghetti, B., Tagliavini, F., Frangione, B., and Prelli, F. (1996) Biochem Biophys Res Commun 223, 572-577).

Analysis of PrP processing in cultured SMB cells (Clarke, M. C., and Haig, D. A. (1970) Nature 225, 100-101), which are persistently infected with scrapie mouse prions, and their uninfected counterparts SMB-PS cells which were cleared of infectivity by chronic pentosan sulfate treatment (Birkett, C. R., Hennion, R. M., Bembridge, D. A., Clarke, M. C., Chree, A., Bruce, M. E., and Bostock, C. J. (2001) Embo J 20, 3351-3358), revealed that proteolytic processing of PrP^{C} and PrP^{Sc} mirrored the processing of PrP^{C} and PrP^{Sc} observed in vivo, with C2 being produced under conditions of prion infection (FIG. 2b). As expected from previous studies of chronically infected cells, levels of PrP27-30 were roughly 10-fold lower in chronically infected cultured cells compared to brain extracts from clinically sick mice.

Two approaches were taken to ensure that the PrP proteolysis observed occurred as a result of specific cellular proteases and not as a result of non-specific proteases acting during extraction. Firstly, treatment of 1.25 μg recombinant MoPrP with glycosidase in the presence or absence of 50 μg total protein from Prnp^{0/0} brain extract did not result in the appearance of cleavage products similar to C1 and C2. Control samples consisted of untreated recombinant mouse PrP and PNGaseF-treated SMB cell lysate. (FIG. 2c). Secondly, the same pattern of full-length, C2 and C1 were observed when SMB detergent extracts were prepared in the presence of 0.1 mM PMSF and protease inhibitor cocktail (Roche Diagnostics Corporation, Indianapolis, Ind.), 0.2 mM MDL28170 or 0.2 mM calpain inhibitor IV compared to control SMB detergent extracts prepared in the absence of inhibitors (FIG. 2d).

Differential Regulation of C1 and C2 Cleavage in the Infected and Uninfected States

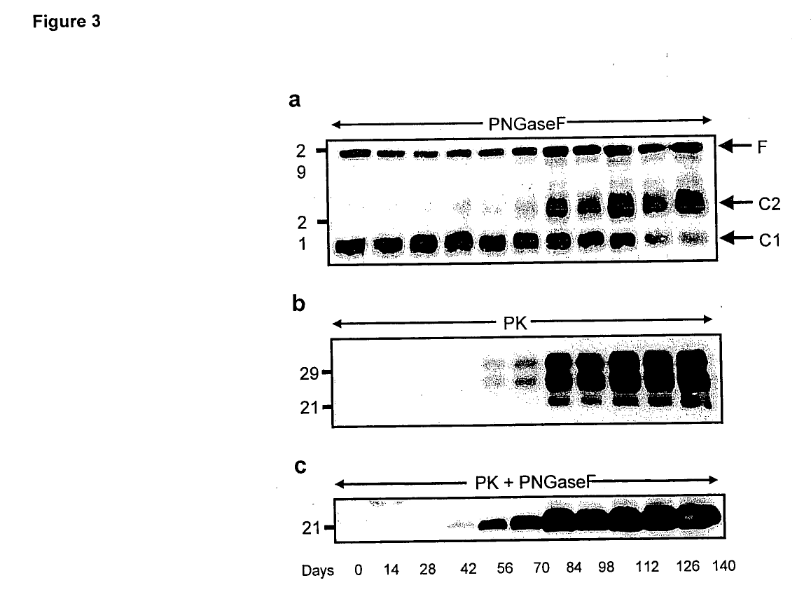

Comparison of C1 and C2 levels in SMB and SMB-PS cells suggested a reciprocal relationship between the two cleavage products in the prion infected and uninfected states (FIG. 2b, lanes 3 and 7). In cured SMB-PS cells where C2 is not produced, C1 levels were 4.7±1.6-fold higher (±SEM, n=3 independent experiments) than in prion infected SMB cells. To more fully characterize the relationship between C1 and C2 during prion infection, the kinetics of C1 and C2 production were analyzed in brains of mice inoculated with mouse-adapted RML scrapie prions. While C2 levels increased between 70 and 84 days post inoculation (FIG. 3a), the presence of cleavage products presumably corresponding to the PK sensitive 21-kDa fragment present at low levels in uninfected mouse brain (FIG. 2a, lane 3), made the exact time of increased C2 production hard to determine. Nonetheless, as C2 levels continued to increase during prion infection, C1 levels correspondingly decreased at the end stage of disease by approximately 3-fold and 4-fold at 126 and 140 days respectively. PrP27-30 was first detected at 56 days (FIG. 3b), with substantial amounts appearing by 84 days. Resolving the various PrP27-30 glycoforms into a single PK-resistant, deglycosylated 21-kDa product resulted in PrP^{Sc} detection in brain extracts as early as 42 days post inoculation (FIG. 3c).

Treatment of Prion-Infected Cells with Cellular Protease Inhibitors Indicating that Production of C2 is Mediated by Calpains

To identify the cellular protease that generates C2 we tested a panel of membrane permeable inhibitors for their ability to affect C2 production in SMB cells. Cells were treated with 20 μM Cathepsin inhibitor III (Z-FG-NHO-BzOME), a cysteine protease inhibitor that selectively inhibits cathepsin B, cathepsin L, cathepsin S and papain; 2 μM Cathepsin L inhibitor III (Z-FY(t-Bu)-DMK) an irreversible inhibitor of cathepsin L; 20 μM Caspase inhibitor III (Boc-D-FMK), a cell-permeable, irreversible, broad-spectrum caspase inhibitor; 2 μM Caspase 3 inhibitor III (Ac-DEVD-CMK) a potent and irreversible inhibitor of caspase-3 as well as caspase-6, caspase-7, caspase-8, and caspase-10; 1 μM of the proteasome inhibitor MG132 (carbobenzoxyl-L-Leucinyl-L-Leucinyl-L-Leucinal-H); 5 μM lactacystin, a highly specific irreversible proteasome inhibitor; 50 μM of the calpain inhibitor MDL28170 (Carbobenzoxy-valinyl-phenylalaninal); 50 μM of the calpain inhibitor calpeptin (Benzyloxycarbonylleucyl-norleucinal); and, 50 μM of calpain inhibitor IV (Z-LLY-FMK) a potent, cell-permeable, and irreversible calpain inhibitor.

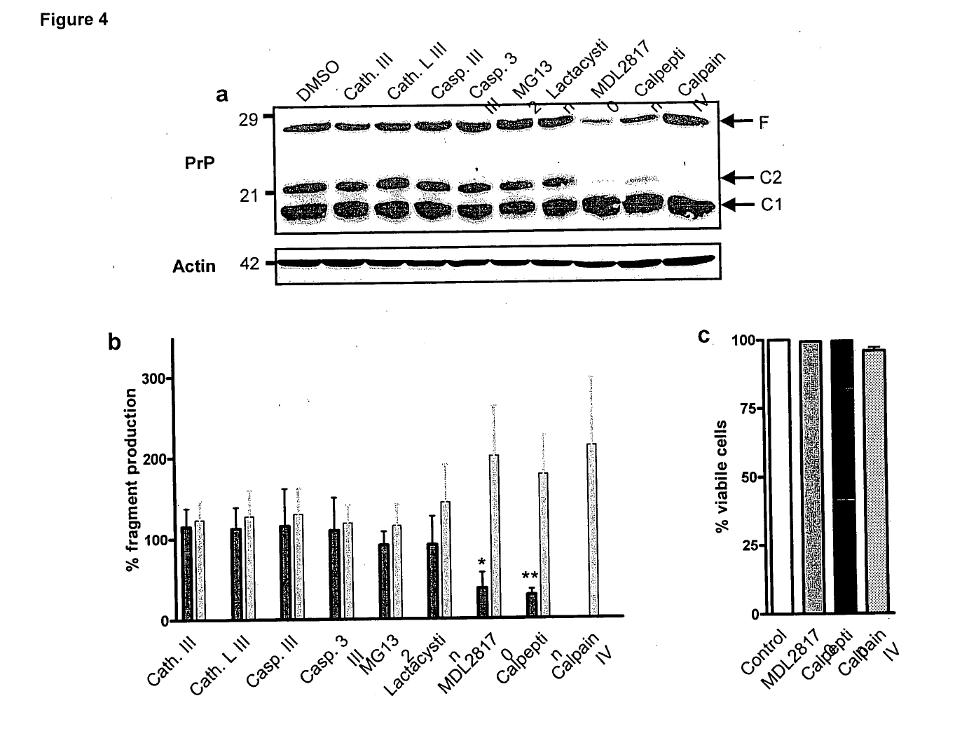

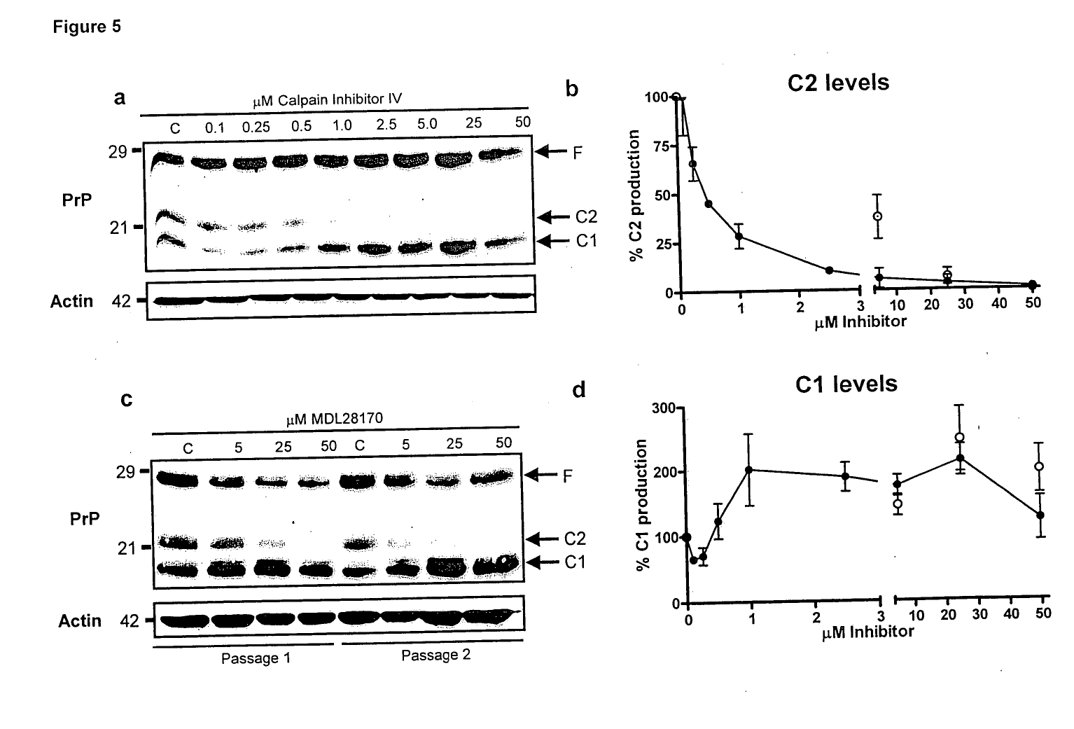

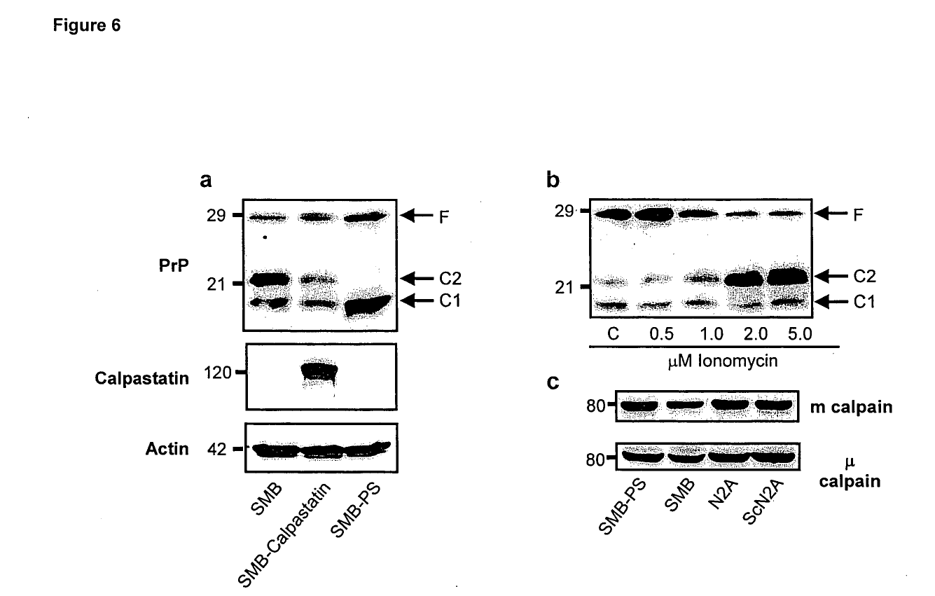

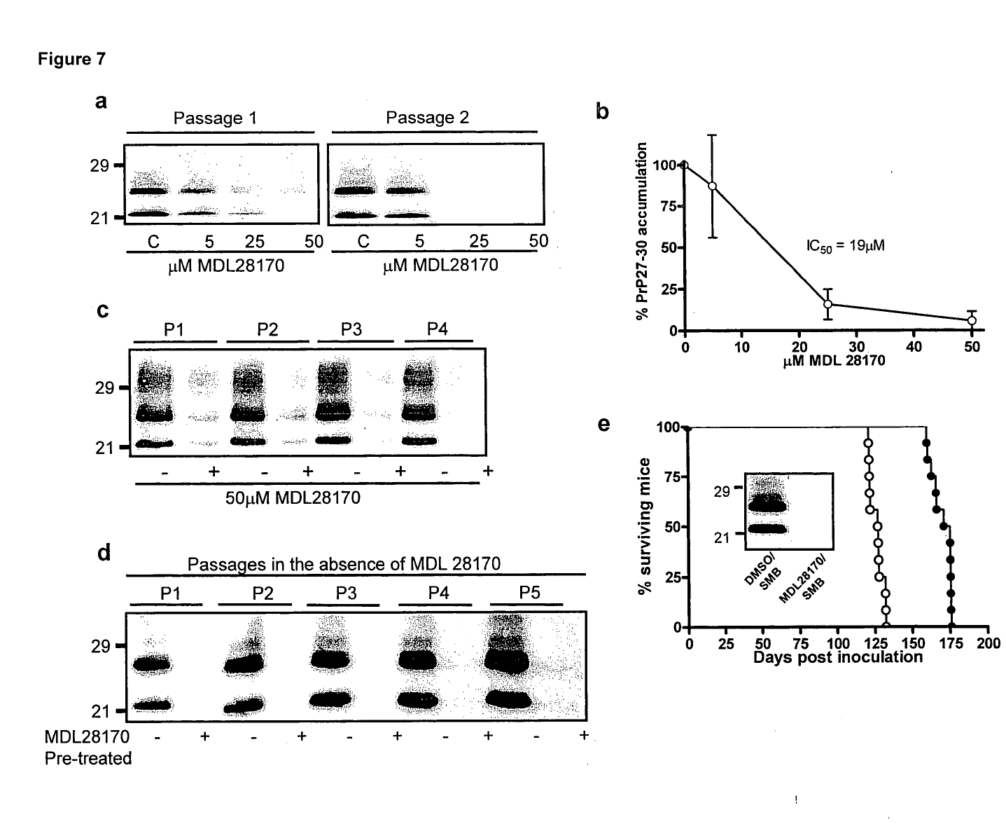

Only calpain inhibitors MDL28170, calpeptin and calpain inhibitor IV inhibited production of C2 while inhibitors of lysosomal proteases, caspases and the proteasome had no effect (FIG. 4a and b). The irreversible calpain inhibitor IV was most effective resulting in apparently complete elimination of C2, while treatment with MDL28170 and calpeptin produced significant reductions in C2, averaging 63±19.7% (±SD, n=3 independent experiments, P=0.031) and 71±7.1% (±SD, n=3 independent experiments, P=0.0033) respectively (FIG. 4b). Since proteolysis by calpains features in a wide range of cellular functions, to ensure that SMB cells could tolerate the maximal concentrations of calpain inhibitors used in these experiments (50 μM) cell viability was monitored (FIG. 4c) and there was no difference in cell survival between the inhibitor-treated and control treated cultures suggesting that reduced C2 production was not the result of a non-specific effect of calpain inhibitors on cell toxicity.

To more fully investigate the effect of calpain inhibitors on PrP processing, SMB cells were cultured in the presence of various concentrations of calpain inhibitor IV or MDL28170. In the case of calpain inhibitor IV treatments, cells were lysed after 4 days when confluent; in the case of MDL28170 treatments, cells were lysed after 4 days when confluent (passage 1) and sub-cultured for a second passage in the presence of the same concentration of MDL28170. Levels of C2 in calpain inhibitor IV-treated SMB cells were reduced in a dose-dependent manner (FIGS. 5a and b). The concentration of calpain inhibitor IV producing 50% inhibition of C2 accumulation (IC₅₀) was calculated to be 0.45 μM. As C2 levels decreased in response to calpain inhibition, C1 correspondingly increased reaching levels ˜2-fold higher than control treated SMB cells at concentrations between 1 μM and 25 μM. At the highest calpain inhibitor IV concentration, C1 levels declined suggesting partial, non-specific inhibition of the protease that cleaves PrP^{C} to produce C1. Production of C2 in MDL28170-treated SMB cells was reduced in a time and dose-dependent manner (FIGS. 5c and d). Treatment for 4 days in the presence of 50 μM MDL28170 resulted in a 75±6.8% (±SD, n=3 independent experiments) reduction in C2, while after 8 days of treatment C2 was undetectable by immunoblotting. The IC₅₀ of MDL28170 was estimated to be 4 μM. Again, as C2 levels decreased in response to MDL28170 at concentrations between 5 μM and 50 μM, C1 levels correspondingly increased to levels similar to calpain inhibitor IV-treated SMB cells (FIG. 5d).

Effects of Calpastatin and Calcium Ionophores on C2 Production

Since calpain activity is tightly regulated in vivo by the intracellular protein inhibitor calpastatin, the unique inhibitory specificity of calpastatin for calpains was exploited by overexpression of calpastatin in SMB cells. Levels of C2 in SMB cells stably overexpressing human calpastatin were 64±15% lower (±SD, n=4 independent experiments) than control transfected cells (P=0.0037) (FIG. 6a).

We also evaluated whether C2 production could be modulated by ionophores that increase intracellular Ca²⁺ with concomitant generation of calpain activity (Guttmann, R. P., and Johnson, G. V. (1998) J Biol Chem 273, 13331-13338). While calpain inhibition abrogated C2 cleavage, treatment of SMB cells for one hour with the Ca²⁺ ionophore ionomycin had the opposite effect, stimulating calpain-mediated cleavage of PrP in the presence of 2 mM Ca²⁺ resulting in a dose-dependent increase in C2 with corresponding reductions in full-length PrP (FIG. 6b). Maximal stimulation of C2 cleavage occurred at 5 μM ionomycin with an average ˜7-fold increase in C2 levels compared to control (n=3 independent experiments). Similarly, treatment with the Ca²⁺ ionophore A23187 at a concentration of 1 μM resulted in 3.5-fold increase of C2 (n=3 independent experiments) (data not shown). We also examined the steady-state levels of m- and μ-calpain in SMB, SMB-PS, N2A and ScN2A cells but found no appreciable differences between prion infected and uninfected cells (FIG. 6c).

Effects of Calpain Inhibition on PrP^{Sc} Accumulation and Prion Titers

Since C2 appears to be predominantly a PrP^{Sc}-specific cleavage product, the effect of calpain inhibition on the accumulation of PrP27-30 was monitored following PK treatment of detergent cell extracts. SMB cells treated with various concentrations of MDL28170 were lysed after 4 days when confluent (passage 1) and sub-cultured for a second passage in the presence of the same concentration of MDL28170. Similar to the effects on C2 levels, treatment of SMB cells with MDL28170 resulted in a time and dose-dependent reduction in the amount of PrP27-30 (FIGS. 7a and b). To determine the effects of MDL28170 in a different cell type persistently infected with mouse-adapted scrapie prions, ScN2A cells (Bosque, P. J., and Prusiner, S. B. (2000) J Virol 74, 4377-4386) were cultured in the presence of 50 μM MDL28170 for varying amounts of time. Accumulation of PrP27-30 was reduced following 4 days of treatment in the presence of MDL28170 (passage 1) and decreased to almost undetectable levels by passage 4 (FIG. 7c).

Since MDL28170 is a reversible calpain inhibitor (Mehdi, S. (1991) Trends Biochem Sci 16, 150-153), an investigation was performed to determine whether PrP^{Sc} production could be reinitiated once calpain activity was restored. Sustained treatment of SMB cells with 50 μM MDL28170 for 5 passages resulted in levels of PrP^{Sc} that were undetectable by immunoblotting. Whereas PrP27-30 was undetectable by immunoblotting for a further 4 passages following removal of the inhibitor and growth in MDL28170-free medium (P1-P4 in FIG. 7d), traces of PrP27-30 were detected at the fifth passage in MDL28170-free medium suggesting a re-emergence of PrP^{Sc} production (P5 in FIG. 7d).

To determine the effects of calpain inhibition on prion replication, bioassays were performed of SMB cells treated with MDL28170 and control treated SMB cells passaged in parallel. After 7 passages (total 33 days) in the presence of 50 μM MDL28170, PrP27-30 was undetectable in treated SMB cells by immunoblotting (inset to FIG. 7e). Inoculation of CD-1 Swiss mice (n=12) with MDL28170-treated SMB cells resulted in a mean incubation time of 170±2 days (±SEM) which was significantly longer (P<0.0001) than the mean incubation time of 126±1.3 days in CD-1 mice (n=12) inoculated with SMB cells treated in parallel with vehicle alone (FIG. 7e). The extended incubation times reflected reduced prion titers in MDL28170-treated SMB cells (Prusiner, S. B., Cochran, S. P., Groth, D. F., Downey, D. E., Bowman, K. A., and Martinez, H. M. (1982) Ann. Neurol. 11, 353-358).

### Discussion

In order to better understand the role of PrP cleavage in prion disease, PrP^{C} and PrP^{Sc} cleavage events were analyzed in brain extracts from prion-inoculated mice and prion-infected cells in culture. Consistent with previous studies (Chen, S. G., Teplow, D. B., Parchi, P., Teller, J. K., Gambetti, P., and Autilio-Gambetti, L. (1995) J. Biol. Chem. 270, 19173-19180, Shmerling, D., Hegyi, I., Fischer, M., Blattler, T., Brandner, S., Gotz, J., Rulicke, T., Flechsig, E., Cozzio, A., von Mering, C., Hangartner, C., Aguzzi, A., and Weissmann, C. (1998) Cell 93, 203-214), it was determined that production of C2 results from endoproteolytic cleavage of PrP^{Sc} by a cellular protease in vivo. A combination of pharmacological and genetical approaches were used to ascertain the nature of the cellular protease responsible for PrP^{Sc} cleavage and to address the role of the C2 cleavage product in the conversion of PrP^{C} to PrP^{Sc} and prion pathogenesis.

The hypothesis that endoproteolytic cleavage of PrP^{Sc} and prion propagation is a calpain dependent process is based on several independent but consistent observations. A panel of membrane permeable protease inhibitors were tested for their ability to hinder the production of C2 in SMB cells. While pharmacological inhibitors of calpains prevented the production of C2, inhibitors of lysosomal proteases, caspases and the proteasome had no effect on C2 production in SMB cells. To address the issue of specificity in our pharmacological studies various inhibitors known were used to specifically target different cellular proteolytic pathways to circumvent the potential for cross-inhibition of cellular proteases. Since most pharmacological inhibitors of calpain also act as weak cathepsin inhibitors, particularly of cathepsin L, there was a concern that the effects we observed might be due to cross inhibition of that system. The demonstration that C2 levels were unaffected by treatment with cathepsin inhibitor III, a selective inhibitor of cathepsins B, L and S and papain, or the more specific, irreversible cathepsin L inhibitor III, suggests that the reductions in C2 levels observed in MDL28170-, calpain inhibitor IV- and calpeptin-treated SMB cells were the result of specific inhibition of the calpain system. Previous studies demonstrating that leupeptin and E-64d affected PrP^{Sc} accumulation in ScN2a cells (Caughey, B., Raymond, G. J., Ernst, D., and Race, R. E. (1991) J. Virol. 65, 6597-6603, Doh-Ura, K., Iwaki, T., and Caughey, B. (2000) J Virol 74, 4894-4897) may be interpreted either in a hypothetical framework involving the control of PrP^{Sc} levels by calpain-dependent and other cysteine protease systems or, we feel more likely, in the context of the known abilities of these broad-spectrum cysteine protease inhibitors to inhibit calpains. Importantly, while calpain inhibitors prevented production of C2, treatment of SMB cells with ionophores that increase intracellular Ca²⁺ with concomitant generation of calpain activity had the opposite effect, resulting in consistent and significant increases in C2 levels. Finally, to substantiate the observation that pharmacological inhibition of calpains prevented cleavage of PrP^{Sc} to produce C2, it was demonstrated that overexpression of the endogenous calpain inhibitor, calpastatin, also affected C2 production in SMB cells. Inhibition of calpains by calpastatin is highly specific and is regarded as the gold standard for demonstrating calpain-dependent cleavage.

It was also found that calpain inhibition prevented PrP^{Sc} accumulation in SMB as well as ScN2A cells and that prion titers in SMB cells were reduced following calpain inhibition. The reappearance of PrP^{Sc} in SMB cells following MDL28170 treatment indicated that, while MDL 28170 effectively inhibited the calpain-mediated cleavage of PrP^{Sc}, the effects of the inhibitor on PrP^{Sc} production were reversible. The apparent absence of PrP^{Sc} by immunoblotting in the MDL 28170-treated SMB inoculum following treatment for 7 passages (inset to FIG. 7e) and the presence of scrapie prions at reduced titers reflect the relative sensitivities of these two assays for prion detection. The ability to reverse the effects of MDL 28170 treatment and observe the re-emergence of PrP^{Sc} in SMB cells demonstrated that this reduction in prion titer corresponded to levels of PrP^{Sc} that were undetectable by immunoblotting but which were sufficient to reinitiate the production and accumulation of additional PrP^{Sc} once calpain activity was restored.

These studies also demonstrated an inverse relationship between the production of the C1 and C2 cleavage products which depended on the state of prion infection in vivo and in SMB cells. As C2 production was eliminated and PrP^{Sc} levels declined in calpain inhibitor treated SMB cells, C1 levels increased to the levels observed in uninfected cells (FIGS. 4 and 5). The possibility that calpain inhibitors indirectly activated the endoproteolytic processing of PrP^{C} resulting in increased C1 production and accompanying down-regulation of C2 was considered. However, since C1 levels were also higher in SMB-PS cells cured of prion infection by pentosan sulfate than in infected SMB cells (FIG. 2b and FIG. 6a) and C1 levels decreased as C2 levels and PrP27-30 increased during prion infection in vivo (FIG. 7), it is believed that the increased levels of C1 subsequent to treatment with calpain inhibitors more likely reflects a conformation-dependent shift from PrP^{Sc} to predominantly PrP^{C} processing as cells change their infected status following inhibition of the C2 cleavage event. Levels of full-length PrP also decreased in response to treatments with calpain inhibitors (FIGS. 4 and 5), again most likely reflecting the shift from PrP^{Sc}/PrP^{C} production in the infected state to only PrP^{C} production in the uninfected state. Calpain inhibition by calpastatin gene transfection was not as efficient or potent as treatment with pharmacological calpain inhibitors. Correspondingly, as C2 was maximally inhibited in calpain inhibitor IV or MDL28170 treated cells and C1 levels increased to the levels observed in uninfected cells, C1 levels remained lower when C2 production was only partially inhibited in SMB cells stably overexpressing human calpastatin.

As suggested by others (Chen, S. G., Teplow, D. B., Parchi, P., Teller, J. K., Gambetti, P., and Autilio-Gambetti, L. (1995) J. Biol. Chem. 270, 19173-19180), conformation-dependent cleavage of PrP^{C} to produce C1 may be a critical determinant in preventing the accumulation of the pathogenic C2 fragment resulting from PrP^{Sc} cleavage at residue 89. Consistent with this notion, epitopes in the region between residues 90 to 120, including the 3F4 binding site which overlaps the C1 cleavage site, were found to be accessible to antibodies in PrP^{C} but largely cryptic in PrP 27-30 (Peretz, D., Williamson, R. A., Matsunaga, Y., Serban, H., Pinilla, C., Bastidas, R. B., Rozenshteyn, R., James, T. L., Houghten, R. A., Cohen, F. E., Prusiner, S. B., and Burton, D. R. (1997) J. Mol. Biol. 273, 614-622). While studies suggest that the PrP^{Sc} conformation may favor cleavage at residue 89 to generate C2, it remains to be determined whether PrP, or specifically PrP^{Sc}, is a calpain substrate. Interestingly, conformational features surrounding cleavage sites in known calpain substrates, particularly when associated with repeated domain elements such as those found in proteins such as tubulin, tau, spectrin and calpastatin, affect calpain substrate sensitivity (Stabach, P. R., Cianci, C. D., Glantz, S. B., Zhang, Z., and Morrow, J. S. (1997) Biochemistry 36, 57-65, Pariat, M., Salvat, C., Bebien, M., Brockly, F., Altieri, E., Carillo, S., Jariel-Encontre, I., and Piechaczyk, M. (2000) Biochem J 345 Pt 1, 129-138, Melloni, E., and Pontremoli, S. (1989) Trends Neurosci 12, 438-444, Johnson, G. V., and Guttmann, R. P. (1997) Bioessays 19, 1011-1018). Cleavage of PrP to produce C2 occurs immediately distal to a tandem array of five octapeptide repeats which are frequently expanded in inherited cases of CJD (FIG. 1). Whether a change in calpain activity and/or calpain redistribution to different subcellular localizations occurs during prion infection also remains to be determined. Since Ca²⁺ modulates calpain activity, the observation that scrapie infection induces abnormalities in Ca²⁺ homeostasis (Kristensson, K., Feuerstein, B., Taraboulos, A., Hyun, W. C., Prusiner, S. B., and DeArmond, S. J. (1993) Neurology 43, 2335-2341) may be significant.

Also relevant in this regard are the findings that treatment of human SH-SY5Y neuroblastoma cells with the neurotoxic PrP106-126 peptide resulted in a rapid rise in intracellular calcium and a concomitant increase in calpain activity (O'Donovan, C. N., Tobin, D., and Cotter, T. G. (2001) J Biol Chem 276, 43516-43523). Interestingly, quinacrine, the most potent substituted tricyclic inhibitor of PrP^{Sc} accumulation (Korth, C., May, B. C., Cohen, F. E., and Prusiner, S. B. (2001) Proc Natl Acad Sci USA 98, 9836-9841), blocks Ca²⁺ channels (Xiao, Y. F., Zeind, A. J., Kaushik, V., Perreault-Micale, C. L., and Morgan, J. P. (2000) Eur J Pharmacol 399, 107-116) raising the intriguing possibility that its mode of action may, at least in part, be related to reducing intracellular Ca²⁺ resulting in lower calpain activity. PrP^{C} to PrP^{Sc} conversion is thought most likely to occur in lipid rafts or in an early endosomal compartment (Caughey, B., Raymond, G. J., Ernst, D., and Race, R. E. (1991) J. Virol. 65, 6597-6603, Vey, M., Pilkuhn, S., Wille, H., Nixon, R., DeArmond, S. J., Smart, E. J., Anderson, R. G., Taraboulos, A., and Prusiner, S. B. (1996) Proc. Natl. Acad. Sci. USA 93, 14945-14949). Increases in intracellular free Ca²⁺ and Ca²⁺-binding to calpain promote translocation of calpains to the plasma membrane (Molinari, M., Anagli, J., and Carafoli, E. (1994) J Biol Chem 269, 27992-27995) and co-localization of m-calpain with detergent-insoluble lipid rafts has been demonstrated in human Jurkat T-cells (Morford, L. A., Forrest, K., Logan, B., Overstreet, L. K., Goebel, J., Brooks, W. H., and Roszman, T. L. (2002) Biochem Biophys Res Commun 295, 540-546). An important unresolved issue is whether calpains adopt a membrane topology that allows direct access to PrP^{Sc} on the cell surface or in the lumen of intracellular vesicles.

The examples set forth above are provided to give those of ordinary skill in the art with a complete disclosure and description of how to make and use the preferred embodiments of the invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference

We claim:

1. A method of treating prion related diseases in a subject in need thereof, comprising administering to the subject a therapeutically effective amount of a calpain inhibitor.

2. The method of claim 1, wherein the calpain inhibitor is selected from the group consisting of small organic molecules, peptides, small interfering RNA's (siRNAs), proteins, and anti-calpain antibodies.
